SEQUENCE LISTING (2 sequences)

SEQ ID NO: 1 - DNA, 24 bp, Artificial Sequence
  PCR primer
acccctggaagcctacaactgcat

SEQ ID NO: 2 - DNA, 24 bp, Artificial Sequence
  PCR primer
gccactgcaccctagcctgtctca

(19) United States
(12) Patent Application Publication
(10) Pub. No.: US 2005/0255506 A1
(43) Pub. Date: Nov. 17, 2005

(54) NOS-2 and glaucoma

(75) Inventor: Claes Wadelius, Uppsala (SE)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Insite Vision Incorporated, Alameda, CA (US)

(21) Appl. No.: 11/059,296

(22) Filed: Feb. 16, 2005

Related U.S. Application Data
(63) Continuation of application No. PCT/US03/26934, filed on Aug. 28, 2003.
(60) Provisional application No. 60/406,993, filed on Aug. 28, 2002.

(51) Int. Cl. C12Q 1/68

(52) U.S. Cl. 435/6

(57) ABSTRACT

Methods of diagnosis of the presence or absence of an NOS-2-associated increased risk of glaucoma, or of a NOS-2-associated decreased risk of glaucoma, are described, in which a sample is tested for the presence of certain alleles of polymorphisms in the promoter of NOS-2, that are associated with an increased risk of glaucoma or with a decreased risk of glaucoma. Also described are methods of therapy of glaucoma, utilizing NOS-2 therapeutic agents.

### RELATED APPLICATIONS

This application is a continuation of International Application No. PCT/US03/26934, which designated the United States and was filed Aug. 28, 2003, published in English, which claims the benefit of U.S. Provisional Application No. 60/406,993, filed Aug. 28, 2002.

The entire teachings of the above applications are incorporated herein by reference.

### BACKGROUND OF THE INVENTION

Glaucoma is a progressive optic neuropathy characterized by a particular pattern of visual field loss and optic nerve head damage resulting from a number of different disorders that affect the eye. Approximately 2.47 million people in the United states are affected with glaucoma (Quigley, H. A. and Vitale, S., Invest. Ophthalmol. Vis. Sci. 38:83 (1997)) and over 100,000 Americans are expected to develop this condition every year. Furthermore, over 67 million people worldwide are estimated to suffer from glaucoma (Quigley, H. A., Br. J. Ophthalmol. 80:389 (1996)). The most common form of this condition is primary open-angle glaucoma (POAG). Glaucomatous optic nerve damage and characteristic visual field loss are the two major clinical signs of this condition (Crick, R. P., Lancet 1:205 (1974); Quigley, H. A., N. Engl. J. Med. 328:1097 (1993); Wilson, R. and Matrone, J. in The Glaucomas, Vol. 2 pp. 753-768 (Ritch, S. M. and Krupin, T., Ed., St. Louis: Mosby, 1996)). Elevated intraocular pressure (IOP) is the most common known risk factor for glaucomatous damage, but it is not equivalent to the disease itself and numerous other risk factors are presently under investigation. Approximately one third to one half of patients with POAG (i.e., up to 1.2 million people in the United States alone) consistently have IOP within the statistically normal range of less than 22 mmHg (Tielsch, J. M. et al., JAMA 266:269 (1991); Hitchings, R. A., Br. J. Ophthalmol. 76:494 (1992); Grosskreutz, C. and Netland, P. A., Int. Ophthalmol. Clin. 34:173 (1994); Werner, E. B. in The Glaucomas, Vol. 2 pp. 768-797 (Ritch, S. M. and Krupin, T., Ed., St. Louis: Mosby, 1996). These patients have been considered to have low- or normal-tension glaucoma (LTG or NTG) and exhibit typical glaucomatous cupping of the optic nerve head and visual field loss (Hitchings, R. A. and Anderton, S. A., Br. J. Ophthalmol. 67:818 (1983)).

During the last decade, eight different genetic loci have been identified for different inherited forms of glaucoma. Two loci have been reported for primary congenital glaucoma (PCG), one for juvenile-onset (JOAG) and another five for adult-onset POAG (Sarfarazi, M. and Stoilov, I., in Ophthalmic Fundamentals: Glaucoma (Sassani, J. W., Ed. (Slack Inc., Thorofare, N.J. 1999), pp. 15-31). However, the causative gene has only been identified for two rare types of this condition, PCG (Stoilov, I. et al., Hum. Mol. Genet. 6:641 (1997)); JOAG (Stone, E. M. et al., Science 275:668 (1997)); and adult-onset POAG (Rezaie, T. et al., Science 295: 1077 (2002)). While ongoing studies show that cytochrome P4501B1 is the major gene for PCG (i.e., 85% of familial and 33% of sporadic cases) (Stoilov, I. et al., Am. J. Hum. Genet. 62:573 (1998)), mutations in the myocilin gene are primarily involved in a small subset of both JOAG and POAG subjects (i.e., 3.0-4.0%), whereas mutations in optineurin may account for 16% of hereditary forms of normal-tension glaucoma. Most of myocilin mutations are identified in JOAG cases (i.e., 2.0-2.5%), though there are other JOAG families that do not have a mutation in this gene (Stoilova, D. et al., J. Med. Genet. 35:989 (1998)). Furthermore, only a handful of mutations are reported in adult-onset POAG cases (i.e., 1.0-1.5%). As yet, no other gene has been identified that is responsible for the adult-onset POAG phenotype.

### SUMMARY OF THE INVENTION

As described herein, a polymorphism in the promoter of a gene encoding inducible nitric oxide synthase (NOS-2), has been associated with risk of glaucoma. Accordingly, the invention pertains to methods of diagnosing the presence or absence of an NOS-2-associated increased risk of glaucoma in an individual, as well as to methods of diagnosing the presence or absence of an NOS-2-associated decreased risk of glaucoma in an individual. The methods include detecting the presence or absence of alleles of a particular polymorphism in the promoter of the NOS-2 gene. The presence of certain alleles of the polymorphism in the promoter is indicative of an NOS-2-associated increased risk of glaucoma. The absence of certain alleles of the polymorphism in the promoter is indicative of the absence of NOS-2-associated increased risk of glaucoma. Similarly, the presence of other different alleles of the polymorphism in the promoter is indicative of an NOS-2-associated decreased risk of glaucoma. The absence of those certain alleles of the polymorphism in the promoter is indicative of the absence of NOS-2-associated decreased risk of glaucoma. The invention additionally pertains to detecting the presence or absence of certain haplotypes; the presence of certain haplotypes is indicative of a NOS-2-associated increased risk of glaucoma, whereas the presence of certain other haplotypes is indicative of a protective effect against NOS-2-associated risk of glaucoma. Similarly, the absence of certain haplotypes is indicative of an absence of a NOS-2-associated increased risk of glaucoma, whereas the absence of certain other haplotypes is indicative of an absence of a protective effect against NOS-2-associated risk of glaucoma

The invention further pertains to methods of treating glaucoma, by administering NOS-2 therapeutic agents, such as nucleic acids encoding NOS-2; agents that alter activity of NOS-2; and/or agents that alter interaction of NOS-2 with NOS-2-interacting polypeptides (e.g., nuclear proteins). Methods of treating glaucoma also comprise use of antisense therapy and homologous recombination. The invention further comprises use of NOS-2 therapeutic agents for the manufacture of a medicament for the treatment of glaucoma.

The methods of the invention allow screening for this disorder in high risk individuals, such as the elderly population, and facilitate both therapeutic and prophylactic treatment for glaucoma.

### DETAILED DESCRIPTION OF THE INVENTION

The present invention relates to a polymorphism in the promoter for the NOS-2 gene, specific alleles of which have been identified as being associated with an increased risk of primary open angle glaucoma, and other specific alleles of which have been identified as being associated with a decreased risk of primary open angle glaucoma. As described herein, Applicant has identified an association between certain alleles of a polymorphism in the promoter of the NOS-2 gene, and an increased risk of glaucoma. Applicant has additionally identified an association between certain alleles of the polymorphism in the promoter of the NOS-2 gene, and a decreased risk of glaucoma. The polymorphism regulates the gene activity by interacting with nuclear proteins.

NOS-2 is an inducible isoform of nitric oxide synthase, that is usually not active, but can be induced by different stimuli like cytokines. Activation of the NOS-2 gene leads to the generation of NO in an amount that is cytotoxic to neighboring cells (Nathan, C. and Xie, Q. W., Cell 78(6):915-8 (1994)). When examined by immunocytochemistry, normal human optic nerve heads are negative for NOS-2. However, NOS-2 was detected in reactive astrocytes of the optic nerve head in glaucoma patients (Liu, B. and Neufeld, A. H., Glia 30(2):178-186 (2000)). Furthermore, no NOS-2 can be detected in primary cell cultures of astrocytes maintained at normal pressure, but in cultures subjected to increased pressure for 1, 2 and 3 days, intense labeling of NOS-2 was shown by immunocytochemistry and immunoblotting. In addition, activation of the NOS-2 gene was shown by semiquantitative PCR of the NOS-2 mRNA (Liu, B. and Neufeld, A. H., Arch Ophthalmol. 119(2):240-5 (2001)).

A highly variable pentanucleotide (CCTTT)n repeat located in an SI hypersensitive region 2.5 kb upstream of the transcription start site, which varies the level of activation of NOS-2 depending on the number of repeats of the pentanucleotide (Warpeha, K. M. et al., FASEB J. 13(13):1825-32 (1999)), was examined. A significant difference was found in the distribution of alleles between cases with simplex glaucoma and matched controls for the CCTTT-repeat (Chi square 18.456, p=0.01, 7DF). Alleles with increased frequency in the glaucoma patients included those having 8, 9, 12, 14, 15 and 16 repeats, whereas alleles with decreased frequency in the glaucoma patients included those having 10 and 13 repeats. Highly significant results for the polymorphism was found when comparing simplex glaucoma and population controls (Chi square 21.167, p=0.0017, 6DF). There was no such significant difference between cases with exfoliative glaucoma and matched or population controls (p=0.09 and 0.11, respectively), and there was no significant difference in the allele distribution between the matched controls and population controls (p=0.3783). When the data from the matched and population controls were pooled and compared to the simplex cases, a highly significant difference was found (Chi square 20.776, p=0.00 12, 7 DF).

Accordingly, the invention pertains to methods of therapy for glaucoma, use of certain compositions for the manufacture of a medicament for use in therapy for glaucoma, and also methods and kits for diagnosing the presence or absence of an increased risk of glaucoma in an individual, or the presence or absence of a decreased risk of glaucoma in an individual, by detecting the presence or absence of certain alleles of polymorphic sites in the NOS-2 promoter. Glaucoma that is associated with the presence of one or more alleles of polymorphic sites in the NOS-2 gene promoter is referred to herein as “NOS-2-associated glaucoma.” An increased risk of glaucoma associated with one or more alleles of polymorphic sites in the NOS-2 gene promoter is referred to herein as “NOS-2-associated increased risk of glaucoma,” and a decreased risk of glaucoma associated with one or more alleles of polymorphic sites in the NOS-2 gene promoter is referred to herein as “NOS-2-associated decreased risk of glaucoma.”

The term, “glaucoma,” as used herein, refers to inherited glaucomas, such as primary congenital or infantile glaucoma; primary open angle glaucoma (POAG), including both juvenile-onset and adult- or late-onset POAG; secondary glaucomas; pigmentary glaucoma; low tension glaucoma (LTG); and normal tension glaucoma (NTG). An “increased risk” of glaucoma, as used herein, refers to a likelihood of an individual for developing glaucoma, that is greater, by an amount that is statistically significant, than the likelihood of another individual or population of individuals for developing glaucoma. A “decreased risk” of glaucoma, as used herein, refers to a likelihood of an individual for developing glaucoma, that is less, by an amount that is statistically significant, than the likelihood of another individual or population of individuals for developing glaucoma.

Methods of Diagnosis and Kits for Diagnosis

In one embodiment of the invention, diagnosis of a NOS-2-associated increased risk of glaucoma, or of a NOS-2-associated decreased risk of glaucoma is made by detecting the presence or absence of particular alleles of one or more polymorphic sites (polymorphisms) in the NOS-2 gene promoter, where the presence of particular alleles (“disease alleles”) is associated with an increased risk of glaucoma and the presence of other particular alleles (“protective alleles”) is associated with a decreased risk of glaucoma. Diagnosis of the presence of a NOS-2-associated increased risk of glaucoma, can be made by identifying the presence of disease alleles directly (e.g., by assessing for the presence of those alleles), or indirectly (e.g., by assessing for the absence of alleles that are not associated with disease); conversely, an absence of a NOS-2-associated increased risk of glaucoma, can be made by identifying the absence of disease alleles directly (e.g., by assessing for the absence of those alleles), or indirectly (e.g., by assessing for the presence of alleles that are not associated with disease). In a similar manner, diagnosis of the presence of a NOS-2-associated decreased risk of glaucoma, can be made by identifying the presence of protective alleles directly (e.g., by assessing for the presence of those alleles), or indirectly (e.g., by assessing for the absence of alleles that are associated with disease); conversely, an absence of a NOS-2-associated decreased risk of glaucoma, can be made by identifying the absence of protective alleles directly (e.g., by assessing for the absence of those alleles), or indirectly (e.g., by assessing for the presence of alleles that are associated with disease).

As used herein, the term, “NOS-2 gene promoter” refers to a nucleic acid at which different proteins including RNA polymerase can bind, to initiate and/or regulate transcription of messenger RNA encoding NOS-2. A “polymorphism,” as used herein, refers to a location in the DNA where different variants in the sequence of nucleic acids are present among individuals in a population; a “polymorphic site” refers to that location in the DNA where the different variants occur. The polymorphism can be a variation in the number of times a unit of base pairs (for example, numbers ranging from one to 50) is repeated in a specific gene (e.g., the CCTTT-repeat described herein); the insertion or deletion of a single nucleotide, or of more than one nucleotide (e.g., an insertion/deletion polymorphism); the change of at least one nucleotide; duplication; transposition; or rearrangement. More than one such polymorphism may be present in the gene promoter.

In a specific embodiment of the invention, the polymorphism is the highly variable pentanucleotide (CCTTT)n repeat 2.5 kb upstream of the transcription start site of NOS-2. A “particular polymorphism” refers to one of the alternate sequences of nucleic acids (for example, one allele): in a preferred embodiment, alleles with 8, 9, 12, 14, 15 and 16 CCCTT-repeats are associated with increased risk of glaucoma (“disease alleles”), and alleles with 10 and 13 repeats are associated with a decreased risk for glaucoma (“protective alleles”). An “allele of interest” refers to a specific allelic variant of the polymorphism (e.g., a disease allele or a protective allele) which is analyzed in the methods described herein.

An “allele associated with an increased risk of glaucoma” refers to a particular allele of the polymorphism, where the allele is found in individuals afflicted with glaucoma, with a frequency that is statistically significantly different (greater) compared to a control population. An “allele associated with a decreased risk of glaucoma” refers to a particular allele of the polymorphism, where the allele is found in individuals afflicted with glaucoma, with a frequency that is statistically significantly different (lower) compared to a control population.

In a first method of diagnosis of an increased or decreased risk of glaucoma, polymerase chain reaction (PCR) amplification and determination of sizes of resulting fragments, are used (see Current Protocols in Molecular Biology, Ausubel, F. et al., eds., John Wiley & Sons, including all supplements through 2002; this document is incorporated herein by reference in its entirety). For example, a test sample of genomic DNA is obtained from an individual, such as, for example, an individual suspected of having, carrying a defect for, or being at increased risk for, glaucoma (the “test individual”). The individual can be an adult, child, or fetus. The test sample can be from any source which contains the nucleic acid, such as a blood sample, serum sample, lymph sample, sample of fluid from the eye (e.g., fluid from the anterior chamber), sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA sample is then examined to determine whether particular alleles of the polymorphism in the NOS-2 gene promoter are present.

For example, amplification by PCR of the sample can be performed using primers (e.g., synthetic oligonucleotides) that specifically hybridize to (e.g., are identical to) sequences on either side of the CCTTT-repeat polymorphism in the NOS-2 gene. Amplification can be used for all, or a portion of the nucleic acid comprising the NOS-2 gene promoter (e.g., the portion that comprises the polymorphic site of the NOS-2 promoter). In a preferred embodiment, primers are selected so that only the sequences of the NOS-2 promoter that are between the primers are specifically amplified. The sizes of the resulting fragments can be determined by standard methods (e.g., electrophoresis in a gel of polyacrylamide, high resolution agarose or high resolution capillary). Sizes of the amplified fragments can be compared to a size standard, and the number of repeats of the two alleles in an individual can be determined. Typically, size standards include an “external size standard,” that is, fragments amplified from a control sample for which the alleles have been characterized in detail, such as by sequence analysis.

The presence of an allele associated with an increased risk of glaucoma (e.g., 14 CCTTT repeats) is diagnostic for an NOS-2-associated increased risk of glaucoma. Similarly, absence of an allele of the polymorphism in the NOS-2 gene promoter that is associated with an increased risk of glaucoma, is diagnostic for the absence of an NOS-2-associated increased risk of glaucoma. The presence of an allele associated with a decreased risk of glaucoma (e.g., 10 or 13 CCTTT repeats) is diagnostic for an NOS-2-associated decreased risk of glaucoma. Similarly, absence of the allele of the polymorphism in the NOS-2 gene promoter that is associated with a decreased risk of glaucoma, is diagnostic for the absence of an NOS-2-associated decreased risk of glaucoma.

In another embodiment, presence or absence of a particular allele of the polymorphism can be indicated by hybridization of the gene promoter (or portion thereof) to a nucleic acid probe. A “nucleic acid probe”, as used herein, is a single-stranded oligonucleotide which hybridizes to the NOS-2 gene promoter. The appropriate length of a probe typically ranges from 15 to 30 nucleotides. Short probes generally require cooler temperatures to form sufficiently stable hybrid complexes with the template. The nucleic acid probe can be a DNA probe or an RNA probe; the nucleic acid probe contains a particular allele of a polymorphism in the NOS-2 gene promoter (e.g., contains a specific number of pentanucleotide repeats in the pentanucleotide CCTTT repeat located 2.5 kb upstream from the transcription start site).

To diagnose the presence or absence of an increased risk of glaucoma or of a decreased risk of glaucoma, a hybridization sample is formed by contacting the test sample containing a NOS-2 gene promoter, with at least one nucleic acid probe. The hybridization sample is maintained under conditions which are sufficient to allow specific hybridization of the nucleic acid probe to the NOS-2 gene promoter. “Specific hybridization”, as used herein, indicates exact hybridization (e.g., with no mismatches). Specific hybridization can be performed under high stringency conditions or moderate stringency conditions, for example.

“Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 95%, 98%). For example, certain high stringency conditions can be used which distinguish perfectly complementary nucleic acids from those of less complementarity.

“High stringency conditions”, “moderate stringency conditions” and “low stringency conditions” for nucleic acid hybridizations are explained on pages 2.10.1-2.10.16 and pages 6.3.1-6 in Current Protocols in Molecular Biology (Ausubel, F. M. et al., “Current Protocols in Molecular Biology”, John Wiley & Sons, (1998), the teachings of which are hereby incorporated by reference). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, high, moderate or low stringency conditions can be determined empirically.

By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology, 200:546-556 (1991). Also, in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of −1720 C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 min at room temperature; a moderate stringency wash can comprise washing in a prewarmed solution (42° C.) solution containing 0.2×SSC/0.1% SDS for 15 min at 42° C.; and a high stringency wash can comprise washing in prewarmed (68° C.) solution containing 0.1×SSC/0.1%SDS for 15 min at 68° C.. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

In a particularly preferred embodiment, the hybridization conditions for specific hybridization are high stringency. Specific hybridization, if present, is then detected using standard methods. If specific hybridization occurs between the nucleic acid probe and the NOS-2 gene promoter in the test sample, then the NOS-2 gene promoter has the particular allele of the polymorphism that is present in the nucleic acid probe. More than one nucleic acid probe can also be used concurrently in this method.

If the nucleic acid probe(s) comprises an allele associated with an increased risk of glaucoma (e.g., 14 CCTTT repeats), specific hybridization of any one of the nucleic acid probes is indicative of the presence of an allele of a polymorphism in the NOS-2 gene promoter that is associated with an increased risk of glaucoma, and is therefore diagnostic for an NOS-2-associated increased risk of glaucoma. Similarly, if the nucleic acid probe(s) comprises an allele of a polymorphism that is associated with increased risk of glaucoma, absence of specific hybridization is indicative of the absence of the allele of the polymorphism in the NOS-2 gene promoter that is associated with an increased risk of glaucoma, and is therefore diagnostic for the absence of an NOS-2-associated increased risk of glaucoma. Conversely, if the nucleic acid probe(s) comprises an allele of a polymorphism that is not associated with increased risk of glaucoma, specific hybridization of any one of the nucleic acid probes is indicative of the absence of an allele of a polymorphism in the NOS-2 gene promoter that is associated with an increased risk of glaucoma, and is therefore diagnostic for the absence of an NOS-2-associated increased risk of glaucoma. Similarly, if the nucleic acid probe(s) comprises an allele of a polymorphism that is not associated with increased risk of glaucoma, absence of specific hybridization is indicative of the presence of an allele of a polymorphism in the NOS-2 gene promoter that is associated with an increased risk of glaucoma, and is therefore diagnostic for the presence of an NOS-2-associated increased risk of glaucoma.

In a similar manner, if the nucleic acid probe(s) comprises an allele associated with a decreased risk of glaucoma (e.g., 10 and/or 13 CCTTT repeats), specific hybridization of any one of the nucleic acid probes is indicative of the presence of an allele of a polymorphism in the NOS-2 gene promoter that is associated with a decreased risk of glaucoma, and is therefore diagnostic for an NOS-2-associated decreased risk of glaucoma. Similarly, if the nucleic acid probe(s) comprises an allele of a polymorphism that is associated with a decreased risk of glaucoma, absence of specific hybridization is indicative of the absence of the allele of the polymorphism in the NOS-2 gene promoter that is associated with a decreased risk of glaucoma, and is therefore diagnostic for the absence of an NOS-2-associated decreased risk of glaucoma. Conversely, if the nucleic acid probe(s) comprises an allele of a polymorphism that is not associated with a decreased risk of glaucoma, specific hybridization of any one of the nucleic acid probes is indicative of the absence of an allele of a polymorphism in the NOS-2 gene promoter that is associated with a decreased risk of glaucoma, and is therefore diagnostic for the absence of an NOS-2-associated decreased risk of glaucoma. Similarly, if the nucleic acid probe(s) comprises an allele of a polymorphism that is not associated with decreased risk of glaucoma, absence of specific hybridization is indicative of the presence of an allele of a polymorphism in the NOS-2 gene promoter that is associated with a decreased risk of glaucoma, and is therefore diagnostic for the presence of an NOS-2-associated decreased risk of glaucoma.

For representative examples of use of nucleic acid probes, see, for example, U.S. Pat. Nos. 5,288,611 and 4,851,330. Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry, 1994, 5, American Chemical Society, p. 1 (1994). The PNA probe can be designed to specifically hybridize to a particular allele of a polymorphism in the NOS-2 gene promoter having an association with glaucoma, or to specifically hybridize to a particular allele of a polymorphism in the NOS-2 gene promoter that does not have an association with glaucoma.

In another method of the invention, analysis by restriction digestion can be used to detect particular alleles of a polymorphism in the NOS-2 gene promoter, if the allele polymorphism results in the creation or elimination of a restriction site. A test sample containing genomic DNA is obtained from the individual. Polymerase chain reaction (PCR) can be used to amplify the NOS-2 gene promoter in the test sample of genomic DNA from the test individual. RFLP analysis is conducted as described (see Current Protocols in Molecular Biology, supra). The digestion pattern of the relevant DNA fragment indicates the presence or absence of particular alleles of the polymorphisms in the NOS-2 gene promoter; a diagnosis of NOS-2-associated increased risk for glaucoma or of NOS-2-associated decreased risk for glaucoma can be made by assessing the presence of alleles associated with increased or decreased risk glaucoma (or, conversely, by assessing the absence of alleles associated with increased or decreased risk of glaucoma).

Sequence analysis can also be used to detect specific alleles of polymorphisms in the NOS-2 gene promoter. A test sample of DNA is obtained from the test individual, as above. PCR or other appropriate methods can be used to amplify the gene promoter, if desired. The sequence of the NOS-2 gene promoter, or a fragment of the gene promoter, is determined, using standard methods. The presence of particular alleles of a polymorphism in the NOS-2 gene promoter (e.g., 13 CCTTT repeats) indicates that the individual has an allele associated with an increased risk of glaucoma, and is therefore diagnostic for an NOS-2-associated increased risk of glaucoma. The absence of a particular allele of a polymorphism in the NOS-2 gene promoter indicates that the individual does not have an allele associated with an increased risk of glaucoma, and is therefore diagnostic for the absence of an NOS-2-associated increased risk of glaucoma. Similarly, the presence of particular alleles of a polymorphism in the NOS-2 gene promoter (e.g., 10 and/or 14 CCTTT repeats) indicates that the individual has an allele associated with a decreased risk of glaucoma, and is therefore diagnostic for an NOS-2-associated decreased risk of glaucoma. The absence of a particular allele of a polymorphism in the NOS-2 gene promoter indicates that the individual does not have an allele associated with a decreased risk of glaucoma, and is therefore diagnostic for the absence of an NOS-2-associated decreased risk of glaucoma.

Allele-specific oligonucleotides can also be used to detect the presence of a particular allele of a polymorphism in the NOS-2 gene promoter, through the use of dot-blot hybridization of amplified oligonucleotides with allele-specific oligonucleotide (ASO) probes (see, for example, Saiki, R. et al., (1986), Nature (London) 324:163-166). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to the NOS-2 gene promoter, and that contains a particular allele of the polymorphism (e.g., an allele associated with glaucoma or with increased risk of glaucoma). An allele-specific oligonucleotide probe that is specific for particular alleles in polymorphisms of the NOS-2 gene promoter can be prepared, using standard methods (see Current Protocols in Molecular Biology, supra). To identify alleles of the polymorphisms that are associated with an increased risk of glaucoma or with a decreased risk of glaucoma, a test sample of DNA is obtained from the individual. PCR can be used to amplify all or a fragment of the NOS-2 gene promoter. The DNA containing the amplified NOS-2 gene promoter (or fragment of the genepromoter ) is dot-blotted, using standard methods (see Current Protocols in Molecular Biology, supra), and the blot is contacted with the oligonucleotide probe. The presence or absence of specific hybridization of the probe to the amplified NOS-2 gene promoter is then detected. Specific hybridization of an allele-specific oligonucleotide probe to DNA from the individual is indicative of the presence of that allele of a polymorphism in the NOS-2 gene promoter, which can then be associated with an increased risk of glaucoma or a decreased risk of glaucoma, as described above.

Other methods of nucleic acid analysis can be used to detect alterations in the NOS-2 gene. Representative methods include direct manual sequencing (Church and Gilbert, (1988), Proc. Natl. Acad. Sci. USA 81:1991-1995; Sanger, F. et al. (1977) Proc. Natl. Acad. Sci. 74:5463-5467; Beavis et al. U.S. Pat. No. 5,288,644); automated fluorescent sequencing; single-stranded conformation alteration assays (SSCP); clamped denaturing gel electrophoresis (CDGE); denaturing gradient gel electrophoresis (DGGE) (Sheffield, V. C. et al. (1989) Proc. Natl. Acad. Sci. USA 86:232-236), mobility shift analysis (Orita, M. et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; chemical mismatch cleavage (CMC) (Cotton et al. (1985) Proc. Natl. Acad. Sci. USA 85:4397-4401); RNase protection assays (Myers, R. M. et al. (1985) Science 230:1242); use of polypeptides which recognize nucleotide mismatches, such as E. coli mutS protein; allele-specific PCR, for example.

The invention additionally pertains to detecting the presence or absence of certain haplotypes; the presence of certain haplotypes is indicative of a NOS-2-associated increased risk of glaucoma, whereas the presence of certain other haplotypes is indicative of a protective effect against NOS-2-associated risk of glaucoma. Similarly, the absence of certain haplotypes is indicative of an absence of a NOS-2-associated increased risk of glaucoma, whereas the absence of certain other haplotypes is indicative of an absence of a protective effect against NOS-2-associated risk of glaucoma. These haplotypes are described further in the Exemplification below.

Kits useful in the methods of diagnosis comprise components useful in any of the methods described herein, including for example, hybridization probes, restriction enzymes (e.g., for RFLP analysis), allele-specific oligonucleotides, means for amplification of nucleic acids comprising the NOS-2 gene promoter, or means for analyzing the nucleic acid sequence of the NOS-2 gene promoter, etc.

Methods of Therapy

The present invention also pertains to methods of treatment (prophylactic and/or therapeutic) for glaucoma or for an increased risk of glaucoma, using an NOS-2 therapeutic agent, as well as to use of NOS-2 therapeutic agents for the manufacture of a medicament for the treatment of glaucoma. The methods can be used not only for individuals diagnosed with glaucoma, or suspected of having an NOS-2-associated increased risk of glaucoma; the methods can also be used for individuals diagnosed with or suspected of having glaucoma or an increased risk of glaucoma other than those associated with NOS-2, as they may similarly be beneficial in such individuals by altering the course of the glaucoma. In one embodiment, of the invention, the NOS-2 therapeutic agent alters (e.g., downregulates) the transcription or translation of NOS-2, or alters the interaction of NOS-2 with nuclear proteins.

A “NOS-2 therapeutic agent” is an agent, used for the treatment of glaucoma, that alters (e.g., enhances or inhibits) polypeptide activity and/or gene expression (e.g., an agonist or antagonist). The therapy is designed to inhibit, alter, replace or supplement activity of NOS-2 in an individual, or to inhibit, alter, replace or supplement activity of a NOS-2 interacting polypeptide in an individual.

A NOS-2 therapeutic agent can alter polypeptide activity or gene expression by a variety of means, such as, for example, by providing additional protein or by upregulating the transcription or translation; by altering posttranslational processing of the polypeptide; by altering transcription of splicing variants; or by altering polypeptide activity, or by altering (upregulating or downregulating) transcription or translation. Other NOS-2 therapeutic agents can target NOS-2-interacting polypeptides, to alter activity or expression of genes encoding NOS-2-interacting polypeptides or of other genes in the pathways in which NOS-2 takes part. For example, as described herein, proteins that bind to the CCTTT-repeat can be used. Nucleic acids, peptides, peptidomimetics, antibodies, or small molecules that inhibit the binding of the activating DNA-binding proteins can be administered, thereby inactivating the gene. Alternatively, in another embodiment of the invention, nucleic acids, peptides, peptidomimetics, antibodies, or small molecules that promote or simulate the binding of inactivating proteins can be administered, thereby inactivating the gene.

More than one NOS-2 therapeutic agent can be used concurrently, if desired. The NOS-2 therapeutic agent(s) are administered in a therapeutically effective amount (i.e., an amount that is sufficient to treat the disease, such as by ameliorating symptoms associated with the disease, preventing or delaying the onset of the disease (e.g., particularly for an individual at increased risk for glaucoma), and/or also lessening the severity or frequency of symptoms of the disease). The amount which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

The term, “treatment” as used herein, refers not only to ameliorating symptoms associated with the disease, but also preventing or delaying the onset of the disease, and also lessening the severity or frequency of symptoms of the disease. Thus, “treatment of glaucoma,” as used herein, refers not only to treatment after appearance of symptoms of glaucoma (therapeutic treatment), but also to prophylactic treatment (prior to appearance of symptoms). Treatment may be particularly beneficial for individuals in whom an increased risk of glaucoma has been identified, as it may delay onset of the disease, or prevent symptoms of the disease entirely. Thus, treatment can be used not only for individuals having glaucoma, but those at risk for developing glaucoma (e.g., those at increased risk for glaucoma, such as those having a polymorphism in the NOS-2 gene promoter that is associated with increased risk of glaucoma).

“Antisense” Therapy

In one embodiment of the invention, a nucleic acid can be used in “antisense” therapy, in which a nucleic acid (e.g., an oligonucleotide) which specifically hybridizes to the DNA of the NOS-2 gene promoter or transcribed region is administered or generated in situ. The antisense nucleic acid that specifically hybridizes to the DNA inhibits expression of the NOS-2 polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct can be delivered, for example, as an expression plasmid as described above. When the plasmid is transcribed in the cell, it produces RNA which is complementary to a portion of the DNA of the NOS-2 gene promoter. Alternatively, the antisense construct can be an oligonucleotide probe which is generated ex vivo and introduced into cells; it then inhibits expression by hybridizing with the genomic DNA. In one embodiment, the oligonucleotide probes are modified oligonucleotides which are resistant to endogenous nucleases, e.g. exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al. ((1988) Biotechniques 6:958-976); and Stein et al. ( (1988) Cancer Res 48:2659-2668). With respect to antisense DNA, oligodeoxyribonucleotides derived from the translation initiation site, e.g. between the −10 and +10 regions of the NOS-2 gene sequence, are preferred.

To perform antisense therapy, oligonucleotides (mRNA, cDNA or DNA) are designed that are complementary to DNA of the NOS-2 gene promoter. The antisense oligonucleotides bind to DNA and prevent transcription. Absolute complementarity, although preferred, is not required. A sequence “complementary” to a portion of DNA, as referred to herein, indicates that a sequence has sufficient complementarity to be able to hybridize with the DNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an DNA it may contain and still form a stable duplex (or triplex, as the case may be). One skilled in the art can ascertain a tolerable degree of mismatch by use of standard procedures.

Nucleic acid molecules to be used in triple helix formation for the inhibition of transcription are preferably single stranded and composed of deoxyribonucleotides. The base composition of these oligonucleotides should promote triple helix formation via Hogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex. The potential sequences that can be targeted for triple helix formation may be increased by creating a “switchback” nucleic acid molecule which is synthesized in an alternating 5′-3′, 3′-5′ manner, such that they base pair with first one strand of a duplex and then the other, eliminating the necessity for a sizable stretch of either purines or pyrimidines to be present on one strand of a duplex.

In a preferred embodiment, oligonucleotides that are complementary to the promoter region (e.g., 5′ end of the message, e.g., the 5′ untranslated sequence up to and including the AUG initiation codon), are used to inhibit translation. However, sequences complementary to the 3′ untranslated sequences of mRNAs have recently been shown to be effective at inhibiting translation of mRNAs as well (Wagner, R. (1994) Nature 372:333); therefore, oligonucleotides complementary to either the 5′ or 3′ untranslated, non-coding regions of the NOS-2 gene can also be used in an antisense approach to inhibit translation of endogenous mRNA. Oligonucleotides complementary to the 5′ untranslated region of the mRNA can include the complement of the AUG start codon. Antisense oligonucleotides complementary to mRNA coding regions can also be used in accordance with the invention. While antisense nucleotides complementary to the translated region can be used, those complementary to the transcribed untranslated region can also be used. Whether designed to hybridize to the 5′, 3′ or coding region of NOS-2 mRNA, antisense nucleic acids are preferably at least six nucleotides in length, and are more preferably oligonucleotides ranging from 6 to about 50 nucleotides in length. In certain preferred embodiments, the oligonucleotide is at least 10 nucleotides, at least 18 nucleotides, at least 24 nucleotides, or at least 50 nucleotides.

If desired, in vitro studies can be performed to quantitate the ability of the antisense oligonucleotide to inhibit gene expression. These studies utilize controls that distinguish between antisense gene inhibition and nonspecific biological effects of oligonucleotides. These studies can compare levels of the target RNA or protein with that of an internal control RNA or protein. In a preferred embodiment, the control oligonucleotide is of approximately the same length as the test oligonucleotide and the nucleotide sequence of the oligonucleotide differs from the antisense sequence on so much so as to prevent specific hybridization to the target sequence.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (see, e.g., Letsinger et al. (1989) Proc. Natl. Acad. Sci. USA 86:6553-6556; Lemaitre et al., (1987), Proc. Natl. Acad Sci. USA 84:648-652; PCT International Publication No. W088/09810) or the blood-brain barrier (see, e.g., PCT International Publication No. W089/10134), or hybridization-triggered cleavage agents (see, e.g., Krol et al. (1988) BioTechniques 6:958-976) or intercalating agents. (See, e.g., Zon, (1988), Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide can comprise at least one (or more) modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. The antisense oligonucleotide can also comprise at least one modified sugar moiety selected from the group including but not limited to arabinose, 2-fluoroarabinose, xylulose, and hexose. In another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof. In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., (1987), Nucl. Acids Res. 15:6625-6641). The oligonucleotide is a 2′-0-methylribonucleotide (Inoue et al. (1987), Nucl. Acids Res. 15:6131-6148), or a chimeric RNA-DNA analogue (Inoue et al. (1987) FEBS Lett. 215:327-330).

Oligonucleotides can be synthesized by standard methods known in the art and described herein (e.g. by use of an automated DNA synthesizer (such as are commercially available from Biosearch, Applied Biosystems, etc.). As examples, phosphorothioate oligonucleotides can be synthesized by the method of Stein et al. ((1988) Nucl. Acids Res. 16:3209), methylphosphonate oligonucleotides can be prepared by use of controlled pore glass polymer supports (Sarin et al, (1988) Proc. Natl. Acad. Sci. USA. 85:7448-7451), etc.

The antisense molecules are delivered to cells which express NOS-2 in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous transcripts and thereby prevent translation of the mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct which can be introduced directly into the tissue site (e.g., the ocular tissue). Alternatively, viral vectors can be used which selectively infect the desired tissue, in which case administration may be accomplished by another route (e.g., systematically).

Homologous Recombination in Therapy

Endogenous NOS-2 gene expression, particularly mutant NOS-2 gene expression or expression of an allele that is associated with an increased risk of disease, can also be reduced by inactivating or “knocking out” the NOS-2 gene promoter, using targeted homologous recombination (e.g., see Smithies et al. (1985) Nature 317:230-234; Thomas & Capecchi (1987) Cell 51:503-512; Thompson et al. (1989) Cell 5:313-321). For example, a non-functional NOS-2 gene (or a completely unrelated DNA sequence) flanked by DNA homologous to the endogenous NOS-2 gene (either the coding regions or regulatory regions of the NOS-2 gene) can be used, with or without a selectable marker and/or a negative selectable marker, to transfect cells that express NOS-2 in vivo. Insertion of the DNA construct, via targeted homologous recombination, results in inactivation of the NOS-2 gene. The recombinant DNA constructs can be directly administered or targeted to the required site in vivo using appropriate vectors, as described above. Alternatively, expression of non-mutant NOS-2 can be increased using a similar method: targeted homologous recombination can be used to insert a DNA construct comprising a non-mutant, functional gene in place of a mutant gene in the cell, as described above. For example, a protective allele can be inserted via homologous recombination, in place of a disease allele. Thus, endogenous NOS-2 gene expression can be altered by targeting deoxyribonucleotide sequences complementary to the regulatory region of the gene (i.e., the promoter and/or enhancers) to form triple helical structures that prevent transcription of the NOS-2 gene in target cells in the body. (See generally, Helene, C. (1991) Anticancer Drug Des., 6(6):569-84; Helene, C., etal. (1992) Ann, N.Y Acad. Sci., 660:27-36; and Maher, L. J. (1992) Bioassays 14(12):807-15).

Agents that Interact with NOS-2

A NOS-2 therapeutic agent can be a polypeptide which interacts with NOS-2; a nucleic acid encoding such a polypeptide which interacts with NOS-2; an agent which alters the expression or activity of NOS-2-interacting polypeptide(s); and/or an agent which alters the interaction between NOS-2 and NOS-2-interacting polypeptide(s). For example, as described below in detail, certain polypeptides bind to the CCTTT-repeat in the NOS-2 promoter. Thus, alteration (increase or decrease) of the expression of any one of these polypeptides which interact with NOS-2 will alter the amount of activity of NOS-2. Agents which alter the expression or activity of NOS-2-interacting polypeptides can be, for example, any of the types of agents described herein (e.g., nucleic acids, polypeptides or proteins, peptidomimetics, antibodies, etc.).

A combination of any of the above methods of treatment, can also be used.

Compositions for Methods of Treatment

The methods of treatment described above utilize agents which can be incorporated into pharmaceutical compositions, if desired. For instance, a nucleotide or nucleic acid construct (vector), can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. In a preferred embodiment, the composition is introduced intraocularly (e.g., eye drops). Other suitable methods of introduction can also include gene therapy (as described below), rechargeable or biodegradable devices, particle acceleration devises (“gene guns”) and slow release polymeric devices. The pharmaceutical compositions can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions that can be used in the methods of treatment. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

The following Exemplification is offered for the purpose of illustrating the present invention and are not to be construed to limit the scope of this invention. The teachings of all references cited herein are hereby incorporated herein by reference in their entirety.

### EXEMPLIFICATION

### Relationship Between Polymorphisms in the NOS-2 Promoter and Glaucoma and Identification of Haplotypes

### Polymorphisms in the NOS-2 Promoter

A PCR method was developed using fluorescently labelled primers and detection on an ABI377 DNA sequencer. Accurate size determination was achieved by an internal size standard. The sizes of the alleles were determined in cases with primary open angle (simplex) glaucoma, and exfoliative glaucoma, in the carefully selected controls, matched for age, sex and ethnicity in whom glaucoma was excluded, and in healthy blood donors (“population controls”). Differences in the number of alleles were evaluated using Chi square analysis.

A highly variable pentanucleotide (CCTTT)n repeat located in an S1 hypersensitive region 2.5 kb upstream of the transcription start site, which varies the level of activation of NOS-2 depending on the number of repeats of the pentanucleotide (Warpeha, K. M. et al., FASEB J. 13(13):1825-32 (1999)), was examined.

Genotyping of the CCTTT-repeat could be performed by different methods. One method is PCR amplification and size determination. Alternative methods (e.g., sequence analysis) generates the same results.

PCR primers were used: forward 5′-(FAM)-ACC CCT GGA AGC CTA CAA CTG CAT-3′ (SEQ ID NO: 1); reverse, 5′-GCC ACT GCA CCC TAG CCT GTC TCA-3′ (SEQ ID NO: 2). The forward primer was labeled with the fluorescent dye FAM for detection on an automated DNA sequencer (ABI 377, 3700 or equivalent). Using these primers, the amplified region was 186 base pairs on an allele (variant) with 10 perfect CCTTT-repeats.

PCR was performed in 10 μl containing 25 ng genomic DNA from the individual to be analyzed, 1.5 pmol of each primer (Genset Oligos, France), 3 nmol of a dNTP mixture (from a stock of 10 mM, Nucleix Plus™, Amersham Pharmacia Biotech Inc.), 1 μl PCR buffer (from a 10× stock containing 15 mM MgCl2 (Roche or Applied Biosystems), and 0.3 U Ampli Taq Gold™ polymerase (Roche or Applied Biosystems) and water to the final volume of 10 μl. The temperature cycling was performed on a PTC-225 MJ Research PCR machine (SDS, Sweden). The PCR protocol employed an initial denaturation at 95° C. for 10 min, followed by 38 cycles of denaturation 95° C. for 30 seconds, annealing at 91° C. for 1 min, and extension at 72° C. for 45 sec, ending with a 7 min additional extension at 72° C.

TAMRA™ Size Standard, GeneScan™-350 (Applied Biosystems) was used as the size marker. The samples were electrophoresed on a 4% denaturing polyacrylamide gel in an ABI Prism™ 377 DNA Sequencer (Applied Biosystems) and data was collected with 377-96 DNA Sequencer Data Collection version 2.5 (Perkin Elmer or Applied Biosystems). The gel image was analyzed with GeneScan® version 3.1 (Perkin Elmer, ABI), and the results with Genotyper® version 2.5 (Perkin Elmer, ABI).

Results of the genotyping are shown in Table 1.

TABLE 1
Distribution of alleles in cases and controls.

| | | | | | | | | Population |
|---|---|---|---|---|---|---|---|---|
| | | Simplex | | Exfolative | | Matched controls | | controls |
| Repeat No | Allele | Total | Freq (%) | Total | Freq (%) | Total | Freq (%) | Total |
| 8 | 176 | 4 | 1 | 5 | 1.27 | 2 | 0.50 | 2 |
| 9 | 181 | 17 | 4.25 | 13 | 3.30 | 9 | 2.25 | 15 |
| 10 | 186 | 37 | 9.25 | 43 | 10.91 | 55 | 13.75 | 58 |
| 11 | 191 | 81 | 20.25 | 67 | 17.01 | 83 | 20.75 | 73 |
| 12 | 196 | 160 | 40 | 159 | 40.36 | 151 | 37.75 | 137 |
| 13 | 201 | 42 | 10.5 | 53 | 13.45 | 65 | 16.25 | 78 |
| 14 | 206 | 41 | 10.25 | 39 | 9.90 | 22 | 5.50 | 33 |
| 15 | 211 | 9 | 2.25 | 10 | 2.54 | 6 | 1.50 | 7 |
| 16 | 216 | 8 | 2 | 5 | 1.27 | 6 | 1.50 | 2 |
| 17 | 221 | 1 | 0.25 | 0 | 0.00 | 1 | 0.25 | 1 |
| | | 400 | | 394 | | 400 | | 406 |

The “Repeat No” identifies the number of CCTTT-repeats.
Allele is the size in base pairs in the assay.

A significant difference was found in the distribution of alleles between cases with simplex glaucoma and matched controls for the CCTTT-repeat (Chi square 18.456, p=0.01, 7DF). Highly significant results were found when comparing simplex glaucoma and population controls (Chi square 21.167, p=0.0017, 6DF). There was no such significant difference between cases with exfoliative glaucoma and matched or population controls (p=0.09 and 0.11, respectively), and there was no significant difference in the allele distribution between the matched controls and population controls (p=0.3783). When the data from the matched and population controls were pooled and compared to the simplex cases, a highly significant difference was found (Chi square 23.791, p=0.0012, 7 DF) (see Table 2).

TABLE 2

Matched + Population
Controls

| Repeat No | Allele | Total | Freq (%) |
|---|---|---|---|
| 8 | 177 | 4 | 0.50 |
| 9 | 182 | 24 | 2.98 |
| 10 | 187 | 113 | 14.02 |
| 11 | 192 | 156 | 19.35 |
| 12 | 197 | 288 | 35.73 |
| 13 | 202 | 143 | 17.74 |
| 14 | 207 | 55 | 6.82 |
| 15 | 212 | 13 | 1.61 |
| 16 | 217 | 8 | 0.99 |
| 17 | 222 | 2 | 0.25 |
| | | 806 | |

Thus, alleles having 8, 9, 12, 14, 15 and 16 CCTTT-repeats were present with increased frequency in the glaucoma patients, whereas alleles having 10 and 13 repeats had decreased frequency in glaucoma patients. The allele having 11 repeats appeared to be neutral.

EMSA

Electrophoresis mobility shift assays (EMSA) were performed in order to identify interaction between protein(s) and the CCTTT-repeat, as evidence that these sequences were responsible for activation of transcription from the NOS2 gene. A doublestranded oligonucleotide containing 10 CCTTT-repeats was incubated with proteins from nuclear extracts from HeLa cells, as follows. The DNA binding assays were performed as described (Hupp, T. R. et al., Cell 71:875-886 (1992)). Radiolabeled double stranded 50-bases long oligonucleotides 5′-(CCTTT)×10-3′ and 5′-(AAAGG)×10-3′ were incubated with nuclear extracts from HeLa cells in standard buffer for 30 minutes. The reactions were then separated on 4% acrylamide gels for 1 h at 200 V at room temperature. The gels were dried and shifted bands identified using a Phosphoimager. Specific and unspecific DNA were used as competitors in the appropriate control experiments.

These experiments revealed interaction with two proteins, one with a relatively stronger and one with a relatively weaker binding to DNA. Similar findings have been made concerning a pentanucleotide repeat in the PIG3 promoter (TGYCC, where Y=C or T), which is polymorphic with 10, 15, 16 or 17 repeats in the population. The tumour suppressor protein p53 binds to this repeat and binding is necessary and sufficient for the transcriptional activation of the PIG3 gene (Contente, A. et al. Nat. Genet. 30:315-320 (2002)). A polymorphic 14 base pair repeat (often referred to as a variable number tandem repeat (VNTR)) in the insulin gene promoter binds to the transcription factor Pur-1 and regulates the transcription (Lew, A. et al., PNAS 97:12508-12512 (2000)) and is associated with fasting insulin levels and predisposition to type 1 diabetes (Bennett, S. T. et al, Nat. Genet. 9:284-292 (1995)). As described herein, it has now been found that the CCTTT-repeat in the NOS2 promoter is associated with glaucoma. The mode of action may be similar to these other similar interactions.

Identification of Haplotypes

Eight SNPs have been identified in the regulatory region of NOS2, from −3.700 bp to +1.600 i.e. in intron 1. Using SNaPshot technology, they were genotyped in the POAG patients and the matched controls. Haplotypes were constructed in the program Phase (Table 2) and their occurrence in the population were verified in nuclear families. Seven were found that are reasonably common in the population and some additional rare ones were also identified. In a Chi Square analysis there is a significant difference in haplotypes between the groups (p=0.036), especially haplotype 6 which varies in frequency and appears to have a protective effect.

Table

| | No in | Frequency | No in | Frequency |
|---|---|---|---|---|
| SNP haplotype | Controls | (%) | Simplex | (%) |
| 1 CCCCGATC | 151 | 38.13 | 154 | 38.89 |
| 2 CCCCGACT | 85 | 21.46 | 85 | 21.46 |
| 3 TCGCTGCC | 64 | 16.16 | 69 | 17.42 |
| 4 CTGTTGCT | 32 | 8.08 | 37 | 9.34 |
| 5 CCCCGACC | 15 | 3.79 | 13 | 3.28 |
| 6 CCGCGGCC | 38 | 9.60 | 17 | 4.29 |
| 7 CTGTTGCC | 3 | 0.76 | 13 | 3.28 |
| 8 Others¹ | 8 | 2.02 | 8 | 2.02 |
| Total no of | 396 | | 396 | |
| haplotypes | | | | |

¹Haplotypes with a frequency of less than 1% in both groups have been added.

The teachings of the references cited herein are incorporated by reference in their entirety.

While this invention has been particularly shown and described with references to preferred embodiments thereof, it will be understood by those skilled in the art that various changes in form and details may be made therein without departing from the spirit and scope of the invention as defined by the appended claims.

1. A method of diagnosing the presence or absence of a NOS-2-associated increased risk of glaucoma in an individual, the method comprising assessing a test sample from the individual for the presence or absence of a disease allele of a polymorphism in the NOS-2 gene promoter, wherein the presence of the disease allele is indicative of the presence of an NOS-2-associated increased risk of glaucoma, and wherein the absence of the allele is indicative of the absence of an NOS-2-associated increased risk of glaucoma.

2. The method of claim 1, wherein the glaucoma is primary open angle glaucoma.

3. The method of claim 1, wherein the disease allele is an allele having (CCTTT)n repeats, and n is selected from the group consisting of: 8, 9, 12, 14, 15 and 16.

4. The method of claim 1, wherein the disease allele is 14 CCTTT-repeats.

5. A method of diagnosing the presence or absence of a NOS-2-associated decreased risk of glaucoma in an individual, the method comprising assessing a test sample from the individual for the presence or absence of a protective allele of a polymorphism in the NOS-2 gene promoter, wherein the presence of the protective allele is indicative of the presence of an NOS-2-associated decreased risk of glaucoma, and wherein the absence of the allele is indicative of the absence of an NOS-2-associated decreased risk of glaucoma.

6. The method of claim 5, wherein the glaucoma is primary open angle glaucoma.

7. The method of claim 5, wherein the protective allele is an allele having (CCTTT)n repeats, and n is selected from the group consisting of: 10 and 13.

8. A method of diagnosing the presence or absence of an NOS-2-associated increased risk of glaucoma in an individual, comprising:
a) obtaining a test sample of nucleic acid comprising all or a portion of NOS-2 gene promoter from the individual,
b) assessing the test sample for the presence or absence of a disease allele of a polymorphism in the NOS-2 gene promoter,
wherein the presence of the disease allele is indicative of an NOS-2-associated increased risk of glaucoma, and the absence of the allele is indicative of the absence of an NOS-2-associated increased risk of glaucoma.

9. The method of claim 8, wherein the assessment of the test sample for the presence or absence of a disease allele of a polymorphism in the NOS-2 gene promoter is performed by allele size determination.

10. The method of claim 8, wherein the assessment of the test sample for the presence or absence of a disease allele of a polymorphism in the NOS-2 gene promoter is performed by direct mutation analysis by restriction digestion.

11. The method of claim 8, wherein the assessment of the test sample for the presence or absence of a disease allele of a polymorphism in the NOS-2 gene promoter is performed by hybridization of a nucleic acid probe to nucleic acid in the test sample from the individual.

12. The method of claim 8, wherein the assessment of the test sample for the presence or absence of a disease allele of a polymorphism in the NOS-2 gene promoter is performed by hybridization of a peptide nucleic acid probe to nucleic acid in the test sample from the individual.

13. The method of claim 8, wherein the assessment of the test sample for the presence or absence of a disease allele of a polymorphism in the NOS-2 gene promoter is performed by sequence analysis of all or a portion of the NOS-2 gene promoter.

14. The method of claim 8, wherein the assessment of the test sample for the presence or absence of a disease allele of a polymorphism in the NOS-2 gene promoter is performed by hybridization of an allele-specific oligonucleotide to nucleic acid in the test sample from the individual.

15. The method of claim 8, wherein the disease allele is an allele having (CCTTT)n repeats, and n is selected from the group consisting of: 8, 9, 12, 14, 15 and 16.

16. The method of claim 8, wherein the disease allele is 14 CCTTT-repeats

17. A method of diagnosing the presence or absence of an NOS-2-associated decreased risk of glaucoma in an individual, comprising:
a) obtaining a test sample of nucleic acid comprising all or a portion of NOS-2 gene promoter from the individual,
b) assessing the test sample for the presence or absence of a protective allele of a polymorphism in the NOS-2 gene promoter,
wherein the presence of the protective allele is indicative of an NOS-2-associated decreased risk of glaucoma, and the absence of the allele is indicative of the absence of an NOS-2-associated decreased risk of glaucoma.

18. The method of claim 17, wherein the assessment of the test sample for the presence or absence of a protective allele of a polymorphism in the NOS-2 gene promoter is performed by allele size determination.

19. The method of claim 17, wherein the assessment of the test sample for the presence or absence of a protective allele of a polymorphism in the NOS-2 gene promoter is performed by direct mutation analysis by restriction digestion.

20. The method of claim 17, wherein the assessment of the test sample for the presence or absence of a protective allele of a polymorphism in the NOS-2 gene promoter is performed by hybridization of a nucleic acid probe to nucleic acid in the test sample from the individual.

21. The method of claim 17, wherein the assessment of the test sample for the presence or absence of a protective allele of a polymorphism in the NOS-2 gene promoter is performed by hybridization of a peptide nucleic acid probe to nucleic acid in the test sample from the individual.

22. The method of claim 17, wherein the assessment of the test sample for the presence or absence of a protective allele of a polymorphism in the NOS-2 gene promoter is performed by sequence analysis of all or a portion of the NOS-2 gene promoter.

23. The method of claim 17, wherein the assessment of the test sample for the presence or absence of a protective allele of a polymorphism in the NOS-2 gene promoter is performed by hybridization of an allele-specific oligonucleotide to nucleic acid in the test sample from the individual.

24. The method of claim 17, wherein the disease allele is an allele having (CCTTT)n repeats, and n is selected from the group consisting of: 10 and 13.

25. A method of treating glaucoma in an individual, comprising administering to the individual an NOS-2 therapeutic agent in a therapeutically effective amount.

26. The method of claim 25, wherein the NOS-2 therapeutic agent alters NOS-2 gene expression.

27. The method of claim 25, wherein the NOS-2 therapeutic agent alters interaction between NOS-2 and a nuclear protein.

28. A method of treating an individual having an increased risk for glaucoma, comprising administering to the individual an NOS-2 therapeutic agent in a therapeutically effective amount.

29. The method of claim 28, wherein the NOS-2 therapeutic agent alters NOS-2 gene expression.

30. The method of claim 28, wherein the NOS-2 therapeutic agent alters interaction between NOS-2 and a nuclear protein.
